(19) United States
(12) Patent Application Publication
(10) Pub. No.: US 2019/0358434 A1
(43) Pub. Date: Nov. 28, 2019

(54) GUIDE EXTENSION CATHETERS AND METHODS FOR USING GUIDE EXTENSION CATHETERS

(71) Applicant: BOSTON SCIENTIFIC SCIMED, INC., MAPLE GROVE, MN (US)

(72) Inventors: JEFFREY STEVEN FULLER, BROOKLYN PARK, MN (US); BRIAN J. HANSON, SHOREVIEW, MN (US); BETH CAMILLERI, SAINT PAUL, MN (US); LOUIS WARNER STEFANICH, MINNEAPOLIS, MN (US)

(73) Assignee: BOSTON SCIENTIFIC SCIMED, INC., MAPLE GROVE, MN (US)

(21) Appl. No.: 16/422,940

(22) Filed: May 24, 2019

Related U.S. Application Data
(60) Provisional application No. 62/676,579, filed on May 25, 2018.

(51) Int. Cl. A61M 25/01; A61B 17/00

(52) U.S. Cl. CPC A61M 25/01; A61M 2025/0063; A61B 17/00234

(57) ABSTRACT

A guide extension catheter may include a proximal shaft and a tubular distal sheath including an exchange channel allowing for access (e.g. side access) into the lumen. Guide extension catheters may be used in conjunction with a guide catheter and a medical device having a distal region disposed within the guide catheter and a proximal region disposed outside of the guide catheter. A portion of the proximal region of the medical device can be passed through the exchange channel and into the lumen, and the guide extension catheter can be advanced into the guide catheter over the medical device without removing the medical device from the guide catheter. The exchange channel can extend in a straight line path along the sheath, or may extend in an indirect path along the sheath.

### CROSS-REFERENCE TO RELATED APPLICATIONS

This application claims the benefit of priority under 35 U.S.C. § 119 of U.S. Provisional Application No. 62/676,579, filed May 25, 2018, the entire disclosure of which is hereby incorporated by reference.

### TECHNICAL FIELD

The present disclosure pertains to medical devices, and methods for manufacturing medical devices. More particularly, the present disclosure pertains to guide extension catheters, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices, for example, guide extension catheters.

An example embodiment includes a guide extension catheter including a proximal shaft having a distal end region, and a distal sheath coupled to the distal end region. The distal sheath includes a sheath wall defining a lumen, a proximal lumen opening, a distal lumen opening, and an exchange channel extending through the sheath wall. The exchange channel extends from the proximal lumen opening to the distal lumen opening, and is designed to allow an access region of a medical device to pass there through and into the lumen, so that the distal sheath can be advanced over the medical device. The exchange channel extends from the proximal lumen opening to the distal lumen opening along an indirect path.

Alternatively or additionally to any of the embodiments above or below, the distal sheath defines a central longitudinal axis, and the sheath wall defines a wall axis that extends between a proximal end of the distal sheath and a distal end of the distal sheath, and the wall axis is parallel to the central longitudinal axis. At least a portion of the exchange channel is non-parallel with the wall axis.

Alternatively or additionally to any of the embodiments above or below, the exchange channel includes at least a portion that is angled, curved, serpentine, sinuous, undulating, winding, slanted, spiral, helical, zigzag, or sine wave shaped.

Alternatively or additionally to any of the embodiments above or below, the distal sheath has a plurality of slots formed therein.

Alternatively or additionally to any of the embodiments above or below, the distal sheath includes closing structure to selectively close the exchange channel.

Alternatively or additionally to any of the embodiments above or below, the distal sheath includes a reinforcing structure.

An example embodiment includes a method of using a guide extension catheter in conjunction with a guide catheter and a medical device. The medical device includes a distal region disposed within the guide catheter and a proximal region disposed outside of the guide catheter. The guide extension catheter includes a proximal shaft and a distal sheath defining a lumen. The distal sheath includes an exchange channel allowing for access to the lumen. The method includes passing a portion of the proximal region of the medical device through the exchange channel and into the lumen; and advancing the guide extension catheter into the guide catheter over the medical device.

Alternatively or additionally to any of the embodiments above or below, the guide catheter is disposed within an anatomy of a patient, and advancing the guide extension catheter into the guide catheter over the medical device includes advancing the guide extension catheter into the anatomy of the patient without removing the distal region of the medical device from the guide catheter.

Alternatively or additionally to any of the embodiments above or below, advancing the guide extension catheter into the guide catheter over the medical device includes advancing the guide extension catheter at least partially into a coronary artery or a peripheral artery.

Alternatively or additionally to any of the embodiments above or below, wherein the distal sheath defines a proximal lumen opening and a distal lumen opening, and the exchange channel extend from the proximal lumen opening to the distal lumen opening.

Alternatively or additionally to any of the embodiments above or below, the exchange channel extends from the proximal lumen opening to the distal lumen opening along a direct straight line.

Alternatively or additionally to any of the embodiments above or below, wherein the exchange channel extends from the proximal lumen opening to the distal lumen opening along a path that is indirect.

Alternatively or additionally to any of the embodiments above or below, wherein the indirect path includes at least a portion that is angled, curved, serpentine, sinuous, undulating, winding, slanted, spiral, helical, zigzag, or sine wave shaped.

An example embodiment includes a guide extension catheter including a proximal shaft having a distal end region, and a distal sheath coupled to the distal end region. The distal sheath has a proximal end and a distal end, defines a lumen extending there through, and has an exchange channel formed therein. The exchange channel extends from the proximal end to the distal end and is designed to allow an access region of a medical device to pass there through and into the lumen so that the distal sheath can be advanced over the medical device. The distal sheath includes a plurality of slots formed therein.

Alternatively or additionally to any of the embodiments above or below, the exchange channel extends from the proximal end to the distal end along a direct straight line.

Alternatively or additionally to any of the embodiments above or below, the exchange channel extends from the proximal end to the distal end along an indirect path.

Alternatively or additionally to any of the embodiments above or below, wherein the indirect path includes at least a portion that is angled, curved, serpentine, sinuous, undulating, winding, slanted, spiral, helical, zigzag, or sine wave shaped.

Alternatively or additionally to any of the embodiments above or below, wherein the proximal shaft has an proximal shaft outer diameter and the distal sheath has a distal sheath outer diameter, and the proximal shaft outer diameter is less than the distal sheath outer diameter.

Alternatively or additionally to any of the embodiments above or below, wherein the proximal shaft has an proximal shaft outer diameter and the distal sheath has a distal sheath inner diameter, and the proximal shaft outer diameter is less than the distal sheath inner diameter.

Alternatively or additionally to any of the embodiments above or below, wherein the proximal shaft has an proximal shaft outer diameter and the lumen has a lumen diameter, and the proximal shaft outer diameter is less than the lumen diameter.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view illustrating an example guide catheter advanced through the aorta to the ostium of a coronary artery;

FIG. 2 is a plan view illustrating an example guide extension catheter used in conjunction with a guide catheter advanced through the aorta to the ostium of a coronary artery;

FIG. 3 is a partial perspective side view of a portion of an example guide extension catheter including an exchange channel;

FIG. 4 is a plan view including the example guide extension catheter from FIG. 3, shown in conjunction with a partial cross-sectional plan view of a guide catheter advanced through the aorta to the ostium of a coronary artery and a treatment catheter advanced into the guide catheter, prior to advancing the guide extension catheter into the guide catheter and/or on or over the treatment catheter;

FIG. 5 is a plan view similar to that shown in FIG. 4, showing the guide extension catheter, including an exchange channel, disposed on or over the treatment catheter prior to advancement into the guide catheter;

FIG. 6 is a plan view similar to that shown in FIG. 5, showing the guide extension catheter, including an exchange channel, disposed on or over the treatment catheter and being advanced into the guide catheter;

FIG. 7 is a partial perspective side view of a portion of another example guide extension catheter including an exchange channel and including slots formed therein;

FIG. 8 is a partial side view of a portion of another example guide extension catheter including an exchange channel and including slots formed therein;

FIG. 9 is a partial side view of a portion of another example guide extension catheter including a differently configured exchange channel and including slots formed therein;

FIG. 10 is a partial side view of a portion of another example guide extension catheter including a differently configured exchange channel;

FIG. 11 is a partial side view of a portion of another example guide extension catheter including a differently configured exchange channel;

FIG. 12 is a partial side view of a portion of another example guide extension catheter including closing structure for selectively/releasably closing the exchange channel;

FIG. 12A is a cross-sectional view of a portion of the guide extension catheter of FIG. 12;

FIG. 12B is a cross-sectional view of another example guide extension catheter similar to that shown in FIG. 12A, but showing a different configuration of a closing structure for selectively/releasably closing the exchange channel;

FIG. 13 is a partial side view of a portion of another example guide extension catheter including an exchange channel and including different structure in the distal sheath;

FIG. 14 is a partial side view of a portion of another example guide extension catheter including a differently configured exchange channel;

FIG. 15 is a partial side view of a portion of another example guide extension catheter including an exchange channel and including different structure in the distal sheath;

FIG. 16A is a partial side view of a portion of another example guide extension catheter, including an expandable distal sheath shown in an expanded configuration; and

FIG. 16B is a partial side view of the example guide extension catheter of FIG. 16A, showing the distal sheath in a contracted and/or non-expanded configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

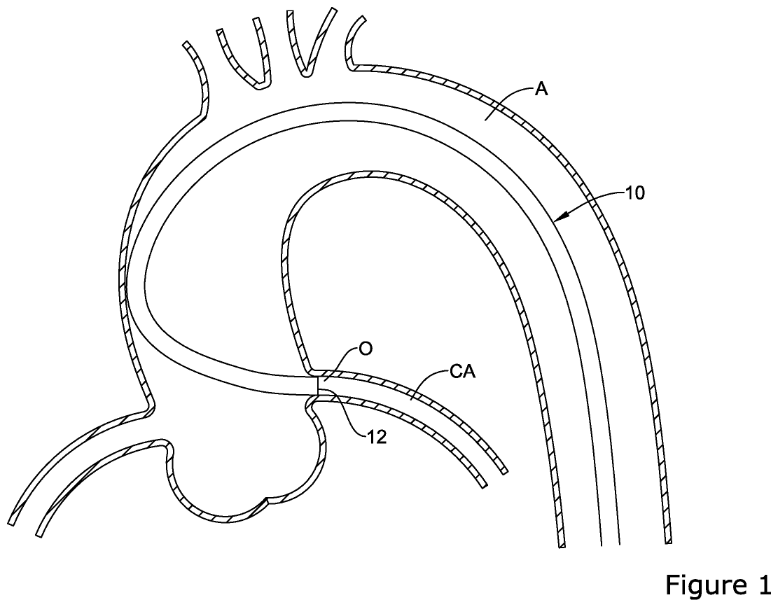

Minimally-invasive cardiac interventions such as percutaneous transluminal coronary angioplasty and/or stent delivery are widely utilized throughout the world. These procedures may include the use of a guide catheter. For example, a guide catheter 10 may be advanced through a blood vessel such as the aorta A to a position adjacent to the ostium O of a (e.g., left and/or right) coronary artery CA as illustrated in FIG. 1. When so positioned, a medical and/or treatment device (e.g., balloon catheter, stent delivery system, guidewire, etc.) may be advanced through the guide catheter 10 and into the coronary artery CA to a target location where the medical and/or treatment device may be used to perform the appropriate cardiac intervention. While the above example shows use of a guide extension catheter for application in accessing a coronary artery, it is also contemplated for use in other anatomy, such as in a peripheral artery.

In order for the medical and/or treatment device to efficiently reach the intended target location, maintaining the position of the guide catheter 10 at the ostium O of the coronary artery CA may be desirable. For example, given that the heart may be beating during the intervention (and/or other factors), the guide catheter 10 may lose its positioning or otherwise be shifted so that it no longer is positioned to efficiently guide the medical and/or treatment device to the coronary arteries. This may include a distal end 12 of guide catheter 10 being shifted away from the ostium O of the coronary artery CA. Because of the shift away from the ostium O, access to the coronary arteries CA may require repositioning of the guide catheter 10 in order to bring the distal end 12 back into engagement with the ostium O of the coronary artery CA.

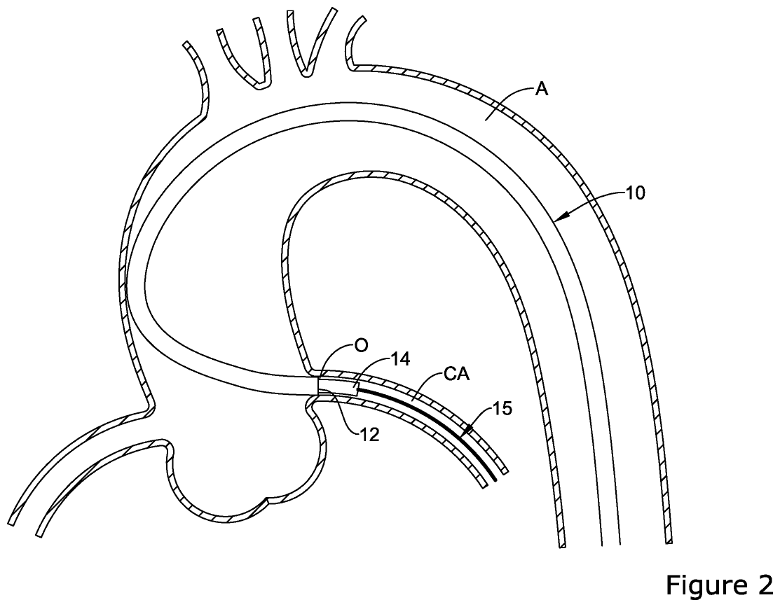

The use of a guide extension catheter may help to improve access to the coronary arteries CA. For example, FIG. 2 illustrates a guide extension catheter 14 extending through the guide catheter 10 and including a portion that extends beyond the distal end 12 of the guide catheter 10 into the coronary artery CA. Because, for example, the guide extension catheter 14 may extend beyond the distal end 12 of the guide catheter 10, the guide extension catheter 14 may extend beyond the ostium O of the coronary artery CA and into a portion of the coronary artery CA. By extending beyond the ostium O, the guide extension catheter 14 may stabilize the positioning of the guide catheter 10 and allow for improved access to the coronary artery CA for a number of cardiac interventions. For example, a medical and/or treatment device 15 may then be advanced through the guide catheter 10 and through the guide extension catheter 14 and into the coronary artery CA to a target location where the medical and/or treatment device 15 may be used to perform the appropriate intervention.

Normally during a procedure, a guide extension catheter needs to be positioned within a guide catheter prior to the introduction of the medical device that will extend there through. In other words, during a procedure, the medical device is normally introduced into the guide catheter after the guide extension catheter is introduced and/or positioned within the guide catheter, so that the medical device can then be fed into, and extend through, the lumen of the guide extension catheter that is within the guide catheter. In many cases, after the medical device has already been introduced into the guide catheter during a procedure, it would be difficult, if not impossible, to then introduce the guide extension catheter into the guide catheter and over the medical device. For example, it may be difficult or impossible to thread the guide extension catheter over and/or onto the very proximal end of the medical device. For example, such medical devices often include a hub and/or manifold disposed at the proximal end thereof, and the guide extension catheter cannot fit there over. Additionally, even if no such hub and/or manifold exists, threading the proximal end of a medical device into a small diameter sheath of a guide extension catheter may be a challenge. This difficulty and/or inability to introduce a guide extension catheter during a procedure and/or after a procedure has begun (e.g. after the medical device is introduced into the guide catheter) may pose some challenges.

For example, in certain situations, a health care professional may initially determine that a guide extension catheter is unwanted and/or unnecessary during a procedure. As such, they proceed without the use of a guide extension catheter, including advancing a guide catheter into the patient, and then advancing a medical device into the guide catheter (e.g. into the anatomy). At some point thereafter during the procedure, the health care professional may determine that a guide extension catheter would be useful and/or desirable, for example, to help stabilize the positioning of the guide catheter and allow for improved access. However, at this point in the procedure, the only way to now introduce a guide extension catheter may be to first remove the medical device from the guide catheter (and/or the anatomy) so that the guide extension catheter may then be introduced into the guide catheter, and only then can the medical device be reintroduced. This may be undesirable.

It would be desirable to provide a guide extension catheter that includes structure and/or a mechanism and/or means for allowing the guide extension catheter to be introduced into the guide catheter and/or around or over the medical device after the medical device has already been introduced into the guide catheter and/or into the anatomy during a procedure. As such, some embodiments of the present disclosure may be so configured.

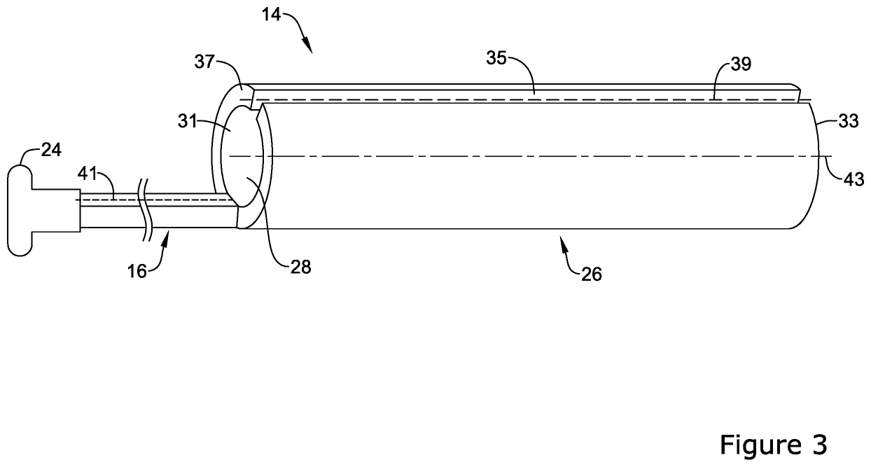

FIG. 3 shows a partial perspective view of a guide extension catheter 14 that includes a proximal shaft or member 16, and a distal sheath 26 defining a lumen 28 and an exchange channel 35 configured for allowing access (e.g. side access) to the lumen 28 there through.

The proximal shaft 16 may be a solid wire or ribbon, or may be a generally tubular member defining a lumen along a part or the entire length thereof, or a combination of these structures. A handle or hub 24 may be attached to proximal shaft 16, for example, near the proximal end of the proximal shaft 16. The proximal shaft 16 may be designed to be sufficiently small (while still being sufficiently sized and configured for pushability) so as to take up relatively little space within the interior of a guide catheter (e.g. guide catheter 10) when a portion thereof is disposed therein. The proximal shaft 16 may be disposed in a side by side relationship with another medical device (e.g. medical device 15) within the lumen of a guide catheter (e.g. guide catheter 10), as discussed herein. The proximal shaft 16 may define and/or extend along a central longitudinal axis 41. The proximal shaft 16 may include or be made of one or more metals, polymers, and/or composite or layered structures thereof, including any of those disclosed herein.

The distal sheath 26 may be attached to proximal shaft 16. For example, a proximal end or region of the distal sheath 26 may be attached to a distal end or region of the proximal shaft 16. The lumen 28 is defined by the distal sheath 26. The distal sheath 26 may define a proximal lumen opening 31 (e.g. at the proximal end of the sheath) and a distal lumen opening 33 (e.g. at the distal end of the sheath), and the lumen 28 may extend through distal sheath 26 from the proximal opening 31 to the distal opening 33. The distal sheath 26 and/or the lumen 28 defined thereby, may define and/or extend along a central longitudinal axis 43. The central longitudinal axis 43 of the distal sheath 26 may be offset from, but parallel to the central longitudinal axis 41 of the proximal shaft 16. The distal sheath 26 includes a sheath wall 37 having a thickness. Further, the sheath wall 37 may define and/or extend along a longitudinally extending wall axis 39, which extends from the proximal opening 31 to the distal opening 33 in a straight line extending through a portion of the wall thickness. The longitudinal wall axis 39 may be parallel to the central longitudinal axis 43 of the distal sheath 26. For example, while the distal sheath 26 may include curved structures and/or surfaces, from a side view, the wall axis 39 will appear as a direct straight line (not angled or curved) extending from the proximal end to the distal end of the sheath 26, as shown. The distal sheath 26 may include or be made of one or more metals, polymers, and/or composite or layered structures thereof, including any of those disclosed herein.

The outer diameter of the distal sheath 26 may be larger than the outer diameter of the proximal shaft 16. Moreover, the outer diameter of the distal sheath 26 may be sufficiently small so as to allow the distal sheath 26 to be passed into and/or disposed within a guide catheter (e.g. guide catheter 10). The inner diameter of distal sheath 26 (e.g. the diameter of the lumen 28) may be larger than the outer diameter of proximal shaft 16. The diameter of the lumen 28 may be sufficiently large so as to allow a medical device (e.g. medical device 15) to pass there through, from the proximal opening 31 to the distal opening 33, for example, along the longitudinal axis 43. As such, when the guide extension catheter 14 is positioned within a guide catheter (e.g. guide catheter 10), a medical device (e.g. medical device 15) may extend within guide catheter alongside the proximal member 16 and through the lumen 28 of the distal sheath 26.

The exchange channel 35 is defined or formed in and/or by the distal sheath 26. The exchange channel 35 may be defined by and/or in the sheath wall 37, and is designed and/or configured to allow for access (e.g. side access) into the lumen 28 from the exterior of the distal sheath 26. The exchange channel 35 extends through the entire thickness of a portion of the sheath wall 37, from the outer surface of the distal sheath 26 to the inner surface of the distal sheath 26. The exchange channel 35 may extend along the distal sheath 26 from the proximal opening 31 (e.g. at the proximal end of the sheath) to the distal opening 33 (e.g. at the distal end of the sheath), and may extend the entire length of the distal sheath 26. The exchange channel 35 may be sized, configured and/or designed to allow a portion of a medical device (e.g. medical device 15) to pass there through so that the medical device can be passed into the lumen 28 through the exchange channel 35. As such, a medical device initially disposed outside of the lumen 28 can be passed through the exchange channel 35 and into the lumen 28, such that distal sheath 26 can be advanced over the medical device. The medical device, once passed through the exchange channel 35 and into the lumen 28, can extend between, and through, the proximal opening 31 and distal opening 33 of the distal sheath 26 within the lumen 28.

The exchange channel 35 may extend along a direct straight line and/or straight path along its length from the proximal opening 31 (e.g. at the proximal end of the sheath) to the distal opening 33 (e.g. at the distal end of the sheath), as shown in FIG. 3. A direct path may be one that does not deviate from a direct straight line between the two end points. For example, the exchange channel 35 may extend parallel to the central longitudinal axis 43 and/or along or parallel to the longitudinally extending wall axis 39. While the distal sheath 26 may include curved structures and/or surfaces, from a side view, the exchange channel 35 may extend along a direct straight line (not angled and/or curved, and does not include any angled or curved portions) along its length, from the proximal end and/or proximal opening 31 to the distal end and/or distal opening 33 of the sheath 26.

In other embodiments, the exchange channel 35, or portions thereof, may extend from the proximal opening 31 (e.g. at the proximal end of the sheath) to the distal opening 33 (e.g. at the distal end of the sheath) along a path that is indirect along at least a portion of its length. An indirect path may be one that includes at least a portion thereof that deviates from a direct straight line between the two end points. In some such embodiments, the exchange channel 35 may be considered to extend in an indirect, or non-straight manner along at least a portion of its length as it extends from the proximal opening 31 to the distal opening 33. For example, the exchange channel 35, or a portion thereof, may be non-parallel to the central longitudinal axis 43 and/or offset from or non-parallel to the longitudinally extending wall axis 39. When viewed from the side, such an indirectly extending exchange channel 35, or portions thereof, may appear as angled, curved, serpentine, sinuous, undulating, winding, slanted, spiraled, helical, zigzag, sine wave shaped, irregular, or the like, or combinations thereof, as it extends along the distal sheath 26 from the proximal opening 31 to the distal opening 33. For example, the tubular-like curvature of the distal sheath 26 will inherently provide an exchange channel 35 that is angled or curved or helically disposed (e.g. relative to the central longitudinal 43 and/or wall axis 39) in the distal sheath 26 with an indirect and/or non-straight line configuration—in that such an exchange channel 35 will follow the tubular curvature of the distal sheath 26. Some example embodiments including such indirect and/or non-straight exchange channels 35 are shown and discussed in other embodiments herein.

In some embodiments, an exchange channel 35 that follows an indirect and/or non-straight path may be desirable so as to better maintain a medical device within the lumen 28 of the guide extension catheter 35 once it is so disposed therein. For example, when the exchange channel 35 follows a path that is non-parallel to the central longitudinal axis 43 and/or the longitudinally extending wall axis 39, it may be less likely that a medical device disposed within the lumen 28 would line up with the exchange channel 35, and inadvertently and/or unintentionally slip out of the exchange channel 35.

The distal sheath 26 may include structure that may allow for the selective opening and/or closing of the exchange channel 35. For example, the distal sheath 26 may include or be made of a resilient material or include structure that may allow for the exchange channel 35 to be opened and/or closed in a “hinging” type motion. For example, the distal sheath 26 may be constructed such that that it is biased so the exchange channel 35 is narrower and/or closed in an initial or relaxed configuration. The exchange channel 35 can then be widened or opened into a second or open configuration, for example with the distal sheath 26 moving in a hinge and/or living hinge like manner, through the application of a predetermined amount of force. In some cases, a user may open the channel by hand, or in other situations, a tool may be used. For example, a tool such as a dilator, trocar, obturator, expandable member, or the like, may be inserted into the distal sheath 26 and used to hold the exchange channel 35 open such that a medical device may be loaded there through, and then the tool may be removed, for example, pulled out axially, to allow the exchange channel 35 to close, thereby containing and/or trapping the medical device within the lumen. The distal sheath 26 may also be configured to revert back to a partially or fully closed position again upon the removal of the predetermined force and/or tool. In some embodiments, the distal sheath 26 may be made of an elastic and/or resilient type material that would allow for this type of movement, while in other embodiments the distal sheath 26 may include a structural hinge or the like.

The distal sheath 26 may include structure and/or material that may prevent and/or make it more difficult for the distal sheath 26 to be undesirably opened too far and/or “splayed open”. For example, the distal sheath 26 may be made of and/or include rigid and/or elastic materials that resist excessive opening of the exchange channel 35. It is also contemplated that the exchange channel 35 may form a path through the distal sheath 26 from the proximal opening 31 to the distal opening 33 that allows the remaining structure of the distal sheath 26 to be interlocking in nature. For example, the pathway of the exchange channel may be undulating such that it forms skinny neck portions and larger head portions that may be interlocking and/or zipper-like structures. Such structures may prevent and/or make it more difficult for the distal sheath 26 to be splayed open.

Furthermore, the distal sheath 26 may include closing structures and/or features to selectively close the exchange channel 35 and/or maintain the distal sheath 26 and/or exchange channel 35 in a selectively closed configuration. Such features and/or structures, allowing for the selective opening/closing of the exchange channel may be desirable so that a medical device may be introduced into the lumen 28 of the guide extension catheter 14 through the exchange channel 35 when desired (e.g. when open), but also so that the exchange channel 35 can be selectively closed to better maintain the medical device within the lumen 28. For example, the distal sheath 26 may include fasteners, latches, magnets, interacting and/or overlapping structures, or the like, disposed along, around and/or about the exchange channel 35 that may be configured to selectively and/or releasably close the exchange channel 35. Some embodiments may include one or a plurality of magnets or pairs of magnets, or magnetic strips disposed along the distal sheath 26 for selectively and/or releasably holding the exchange channel 35 in a closed configuration. In some examples, the distal sheath 26 may include overlapping edge portions that define and/or are disposed along the edge of the exchange channel 35. The distal sheath 26 overlapping portions may include engagement and/or interlocking structures that may be shaped and/or configured to engage each other and selectively and/or releasably hold the exchange channel 35 closed. For example, the overlapping portions from either side of the exchange channel 35 may be curved and/or wrapped about each other to define interlocking edges and/or channels, and/or may define a plurality of wraps in a spiral like fashion, or the like. Some examples of such structures are shown and discussed in some embodiments herein.

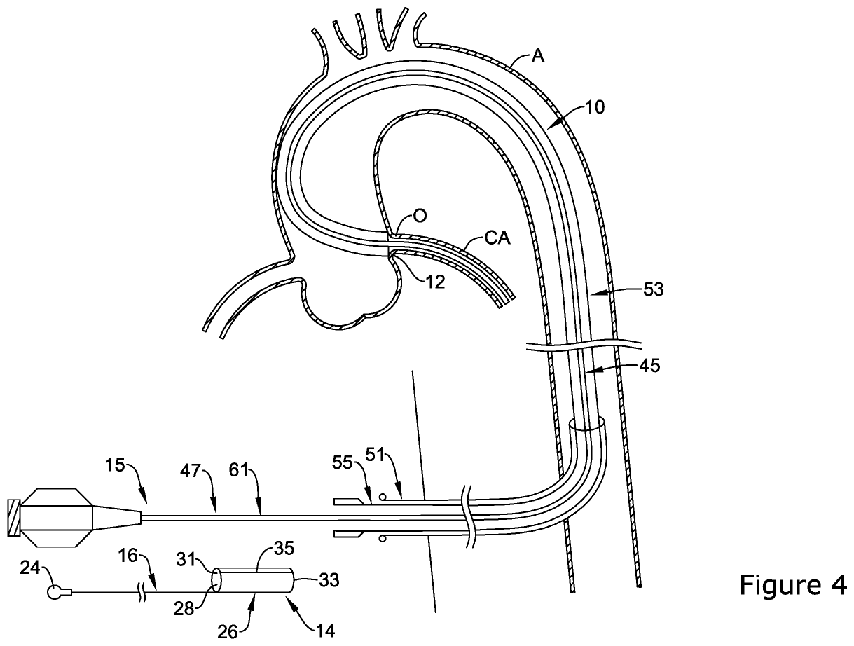

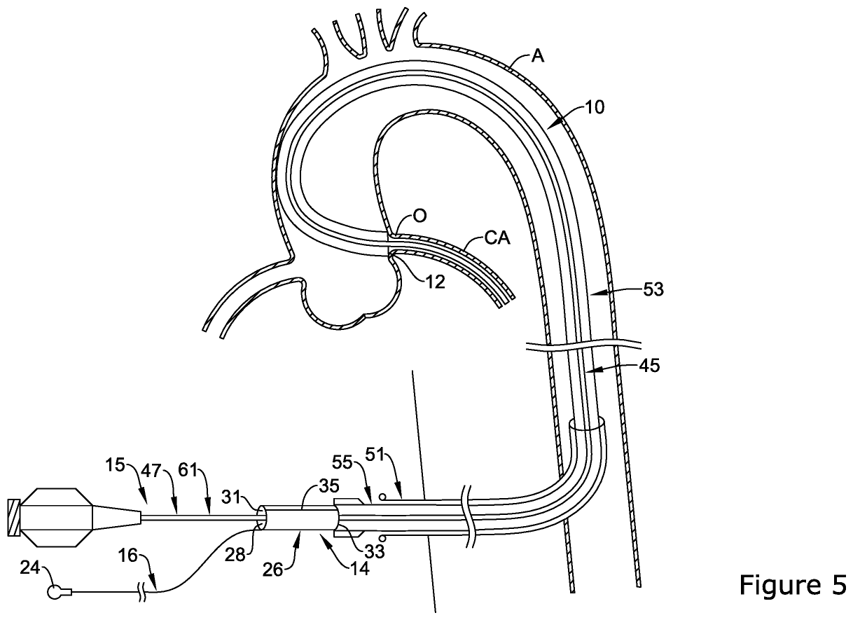

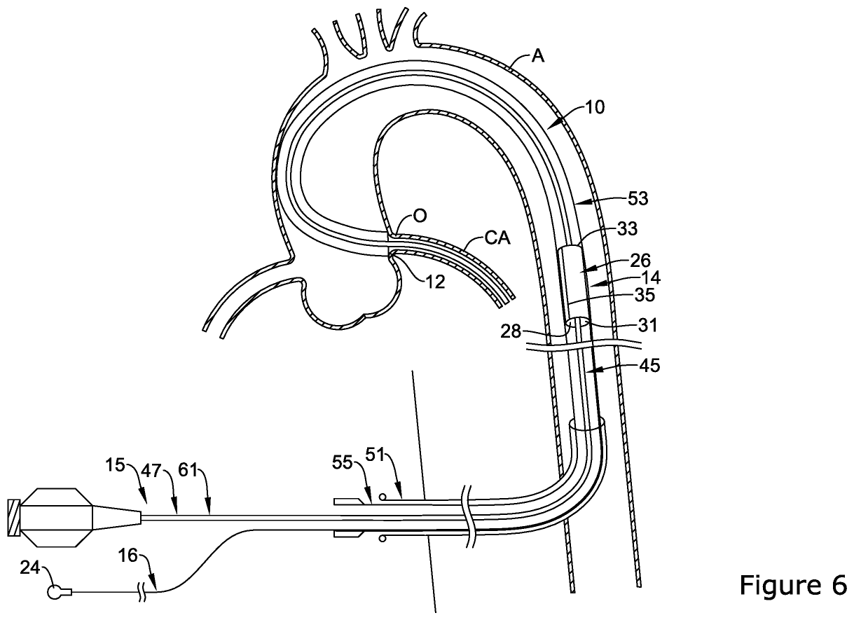

As indicated herein, the guide extension catheter 14 may be configured to be introduced into a guide catheter and/or around or over a medical device after the medical device has already been introduced into the guide catheter and/or into the anatomy during a procedure, without the need to remove the medical device from the guide catheter and/or the anatomy. With reference to FIGS. 4-6, an example method may include using the guide extension catheter 14 in conjunction with a guide catheter 10 and a medical device 15 that includes a distal region 45 disposed within the guide catheter 10 and a proximal region 47 disposed outside of the guide catheter 10; passing a portion of the proximal region 47 of the medical device 15 through the exchange channel 35 and into the lumen 28 (see FIG. 5); and advancing the guide extension catheter 14 into the guide catheter 10 over the medical device 15 (see FIG. 6).

For example, FIG. 4 is a plan view including a partial cross-sectional view of a guide catheter 10 and medical device 15 advanced into an anatomy of a patient. The guide catheter 10 is shown including a distal region 53 advanced into the anatomy, for example through an access sheath 51 and into the vasculature, and advanced into and through the aorta A to the ostium O of a coronary artery CA. A proximal region 55 of the guide catheter 10 remains outside of the anatomy. The medical device 15 (e.g. treatment catheter) is shown advanced into the guide catheter 10 and/or into the anatomy. The medical device 15 includes a distal region 45 disposed within the guide catheter 10 (and largely within the anatomy) and a proximal region 47 disposed outside of the guide catheter 10 (and outside of the anatomy). Also shown in FIG. 4 is a partial perspective side view of the guide extension catheter 14 disposed outside of the guide catheter 10 and/or outside of the anatomy and/or not disposed on or over the medical device 15. The medical device 15 may define an access region 61 that is sized and/or configured to pass through the exchange channel 35 so that the distal sheath 26 can be disposed over and advanced over the medical device 15. While all or any portion of the length of the medical device 15 may be sized and/or configured to pass through the exchange channel 35 and may act as the access region 61, the access region 61 is typically included on the proximal region 47 of the medical device 15 that is disposed outside of the guide catheter 10, but often spaced from the very proximal end of the medical device 15 (which may often include a hub and/or manifold).

FIG. 5 then shows a proximal region 47 (e.g. access region 61) of the medical device 15 passed through the exchange channel 35 and into the lumen 28 of the distal sheath 26. The medical device 15, once passed through the exchange channel 35 and into the lumen 28, can include a portion that extends between, and through, the proximal opening 31 and distal opening 33 of the distal sheath 26 within the lumen 28. As can be appreciated, due to the use of the exchange channel 35, this can be achieved without removing the medical device 15 from the guide catheter 10 and/or the anatomy.

FIG. 6 then shows advancing the guide extension catheter 14 into the guide catheter 10 over the medical device 15 and/or into the anatomy. When the guide extension catheter 14 is positioned within a guide catheter 10, the medical device 15 may extend within guide catheter 10 alongside the proximal member 16 and through the lumen 28 of the distal sheath 26 of the guide extension catheter 14. The guide extension catheter 14 can be advanced further through the guide catheter 10 such that a portion extends beyond the distal end 12 of the guide catheter 10 into the coronary artery CA, as shown in FIG. 2. As discussed herein, the guide extension catheter 14 may extend beyond the ostium O of the coronary artery CA and into a portion of the coronary artery CA, and may help stabilize the positioning of the guide catheter 10 and allow for improved access to the coronary artery CA. The medical device 15 may be disposed and/or delivered there through.

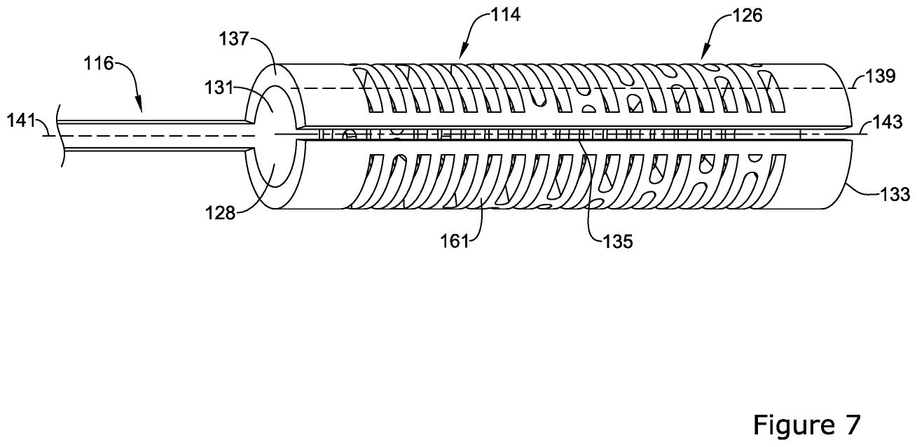

FIGS. 7-16 show some additional example embodiments of guide extension catheters in accordance with this disclosure that may be the same or similar in form and function to the guide extension catheter 14. These example guide extension catheters include a proximal shaft or member, a distal sheath having a wall thickness and defining a lumen and proximal and distal openings into the lumen, and an exchange channel allowing for access (e.g. side access) into the lumen by a medical device. These structures may include structure, materials, and/or usage that are the same as or similar to those of any such similarly named structures shown and described herein. Features, structures, materials, and usage of the various embodiments may be combined and/or used in other embodiments. For example, the discussion of the structure, materials, usage, form and/or function of the guide extension catheter 14, and any related structures in the embodiment above with reference to FIGS. 1-6 may apply equally to any and all of the other guide extension catheter embodiments described herein, and vice versa. In particular, the distal sheath and exchange channel in each of these embodiments, while possibly including some structural differences as noted, may still allow for access to the lumen of the guide extension catheter in a similar manner and in accordance with the methods as described herein.

FIG. 7 shows a partial perspective view of another example embodiment of a guide extension catheter 114 similar in form and function to guide extension catheter 14, but this embodiment includes a plurality of slots 161 defined in the distal sheath 126, as will be discussed in more detail below. The guide extension catheter 114 includes a proximal shaft or member 116, a distal sheath 126 defining a lumen 128 and an exchange channel 135 defined in and extending through a sheath wall 137 and configured for allowing access (e.g. side access) to the lumen 128 there through. The distal sheath 126 may define a proximal opening 131 and a distal opening 133, and the lumen 128 extends from the proximal opening 131 to the distal opening 133 along the length of the sheath 126. The proximal shaft 116 may extend along a central longitudinal axis 141, and the distal sheath 126 and/or lumen 128 may extend along a central longitudinal axis 143. The central longitudinal axis 143 of the distal sheath 126 may be offset from, but parallel to the central longitudinal axis 141 of the proximal shaft 116. Further, the sheath wall 137 may define and/or extend along a longitudinally extending wall axis 139, which extends from the proximal opening 131 to the distal opening 133 in a straight line extending through a portion of the wall thickness. The longitudinal wall axis 139 may be parallel to the central longitudinal axis 143 of the distal sheath 126.

This is another example embodiment where the exchange channel 135 extends along a direct straight line and/or straight path along its length from the proximal opening 131 (e.g. at the proximal end of the sheath) to the distal opening 133 (e.g. at the distal end of the sheath). For example, the exchange channel 135 may extend parallel to the central longitudinal axis 143 and/or along or parallel to the longitudinally extending wall axis 139.

The exchange channel 135 is configured for allowing access (e.g. side access) into the lumen 128 by a medical device. The guide extension catheter 114 can thus be used in a similar manner and/or method to that discussed herein, for example, with reference to FIGS. 3-6, wherein a portion of a medical device may be passed through the exchange channel 135 and into the lumen 128, and the guide extension catheter 114 can be advanced into a guide catheter 10 and over the medical device 15.

As indicated above, the distal sheath 126 includes a plurality of slots 161 formed therein. Various embodiments of arrangements and configurations of the slots 161 are contemplated. In some embodiments, at least some, if not all of the slots 161 are disposed at the same or a similar angle with respect to the longitudinal axis 143 of the distal sheath 126. As shown, the slots 161 can be disposed at an angle that is perpendicular, or substantially perpendicular, and/or can be characterized as being disposed in a plane that is normal to the longitudinal axis 161 of the distal sheath 126. However, in other embodiments, the slots 161 can be disposed at an angle that is not perpendicular, and/or can be characterized as being disposed in a plane that is not normal to the longitudinal axis 143 of the distal sheath 126. Additionally, a group of one or more slots 161 may be disposed at different angles relative to another group of one or more slots 161.

The slots 161 may be provided to enhance the flexibility of distal sheath 126 while still allowing for suitable torque transmission characteristics. The slots 161 may be formed such that one or more rings and/or turns interconnected by one or more segments and/or beams are formed in the distal sheath 126, and such rings and beams may include portions of distal sheath 126 that remain after the slots 161 are formed in the body of the distal sheath 126. Such an interconnected ring structure may act to maintain a relatively high degree of torsional stiffness, while maintaining a desired level of lateral flexibility. In some embodiments, some adjacent slots 161 can be formed such that they include portions that overlap with each other about the circumference of the distal sheath 126. In other embodiments, some adjacent slots 161 can be disposed such that they do not necessarily overlap with each other, but are disposed in a pattern that provides the desired degree of lateral flexibility.

Additionally, the slots 161 can be arranged along the length of, or about the circumference of, the distal sheath 126 to achieve desired properties. For example, adjacent slots 161 can be arranged in a symmetrical pattern, such as being disposed essentially equally on opposite sides about the circumference of the distal sheath 126, or can be rotated by an angle relative to each other about the axis of the distal sheath 126. Additionally, adjacent slots 161, may be equally spaced along the length of the distal sheath 126, or can be arranged in an increasing or decreasing density pattern, or can be arranged in a non-symmetric or irregular pattern. This may include the slots 161 that form or otherwise follow a helical pattern about the distal sheath 126. Other characteristics, such as slot size, slot shape and/or slot angle with respect to the longitudinal axis of the distal sheath 126, can also be varied along the length of the distal sheath 126 in order to vary the flexibility or other properties. In other embodiments, moreover, it is contemplated that portions of the distal sheath 126, such as a proximal section, or a distal section, or the distal sheath 126 as a whole, may not include any such slots 161. In FIG. 7, the slots 161 are shown as being separate from and/or not in communication with and/or are isolated from the exchange channel 135. In other embodiments, however, at least some of the slots 161 may be connected to and/or in communication with and/or included as part of the exchange channel 135.

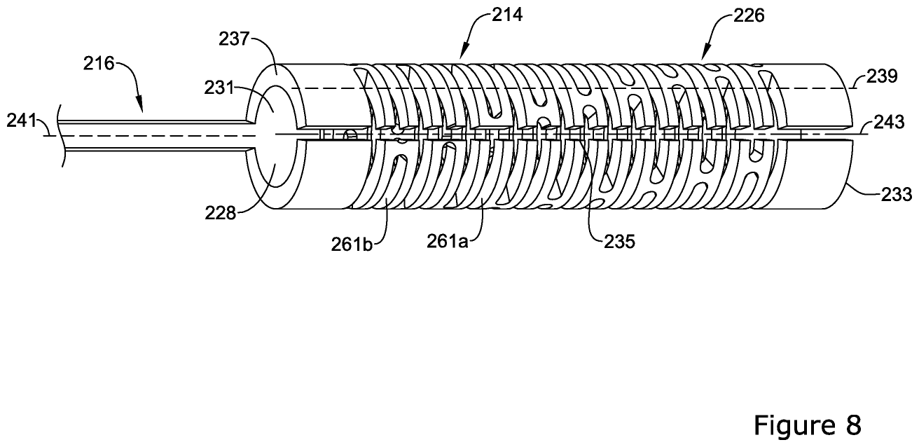

For example, FIG. 8 shows a partial perspective view of another example embodiment of a guide extension catheter 214, which may be similar in form and function to the guide extension catheters 14 and 114. The guide extension catheter 214 in this embodiment includes slots 261a and 261b defined in the distal sheath 226, similar to the slots 161 in the embodiment shown in FIG. 7, but wherein some of the slots 261b are in communication with and/or intersect with and/or are connected to the exchange channel 235. The slots 261a and 261b may be otherwise configured the same or similarly to the slots 161 discussed above relative to FIG. 7.

The remainder of the guide extension catheter 214 may be the same or similar to the other embodiments described herein, and may include structure and/or materials that are the same as or similar to those of any such similarly named structures shown and described herein. For example, the guide extension catheter 214 includes a proximal shaft 216, a distal sheath 226, a sheath wall 237, a lumen 228, a proximal opening 231, a distal opening 233, a proximal shaft central longitudinal axis 241, a distal sheath and/or lumen central longitudinal axis 243, a longitudinally extending wall axis 239, and an exchange channel 235 defined in and extending through the sheath wall 237 and configured for allowing access (e.g. side access) to the lumen 228 there through.

This is another example embodiment where the exchange channel 235 extends along a direct straight line and/or straight path along its length from the proximal opening 231 (e.g. at the proximal end of the sheath) to the distal opening 233 (e.g. at the distal end of the sheath). For example, the exchange channel 235 may extend parallel to the central longitudinal axis 243 and/or along or parallel to the longitudinally extending wall axis 239. The exchange channel 135 is configured for allowing access (e.g. side access) into the lumen 128 by a medical device. The guide extension catheter 114 can thus be used in a similar manner and/or method to that discussed herein, for example, with reference to FIGS. 3-6, wherein a portion of a medical device may be passed through the exchange channel 135 and into the lumen 128, and the guide extension catheter 114 can be advanced into a guide catheter 10 and over the medical device 15.

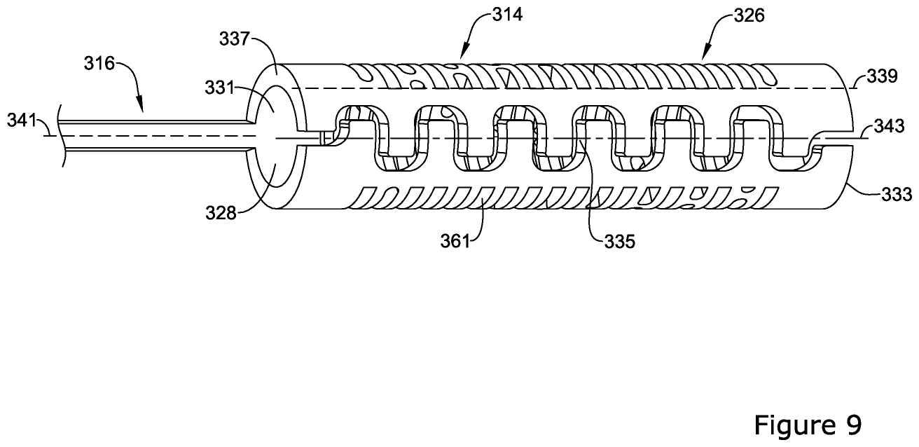

FIG. 9 shows a partial perspective view of another example embodiment of a guide extension catheter 314. The guide extension catheter may be similar in form and function to other embodiments described herein, and may include structure and/or materials that are the same as or similar to other embodiments described herein. For example, similar to other embodiments discussed herein, the guide extension catheter 314 includes a proximal shaft 316, a distal sheath 326, a sheath wall 337, a lumen 328, a proximal opening 331, a distal opening 333, a proximal shaft central longitudinal axis 341, a distal sheath and/or lumen central longitudinal axis 343, a longitudinally extending wall axis 339, and an exchange channel 335 defined in and extending through a sheath wall 337 and configured for allowing access (e.g. side access) to the lumen 328 there through. Similar to the embodiment of FIG. 7, one or more slots 361 may be defined in the distal sheath 326, and the slots 361 may be configured the same or similarly to the slots 161 discussed above relative to FIG. 7 or 8. It is also contemplated that in other similar configurations, the slots may be absent.

As can be appreciated, this embodiment includes an example of an exchange channel 335 that extends along a path that is indirect along at least a portion of its length, as discussed herein. In this particular embodiment, when viewed from the side, the exchange channel 335 follows a generally sinusoidal wave shaped path as it extends along the distal sheath 326 from the proximal opening 331 to the distal opening 333. This exchange channel 335 may be considered to extend in an indirect, or non-straight manner as it extends from the proximal opening 331 to the distal opening 333. For example, the exchange channel 335, or a portion thereof, may be characterized as being non-parallel to the central longitudinal axis 343 and/or non-parallel to the longitudinally extending wall axis 339.

The exchange channel 335 is configured for allowing access (e.g. side access) into the lumen 328 by a medical device. The guide extension catheter 314 can thus be used in a similar manner and/or method to that discussed herein, for example, with reference to FIGS. 3-6, wherein a portion of a medical device 15 may be passed through the exchange channel 335 and into the lumen 328, and the guide extension catheter 314 can be advanced into a guide catheter 10 and over the medical device 15. The medical device 15 would simply be passed through the non-straight (e.g. sinusoidal wave shaped) exchange channel 335.

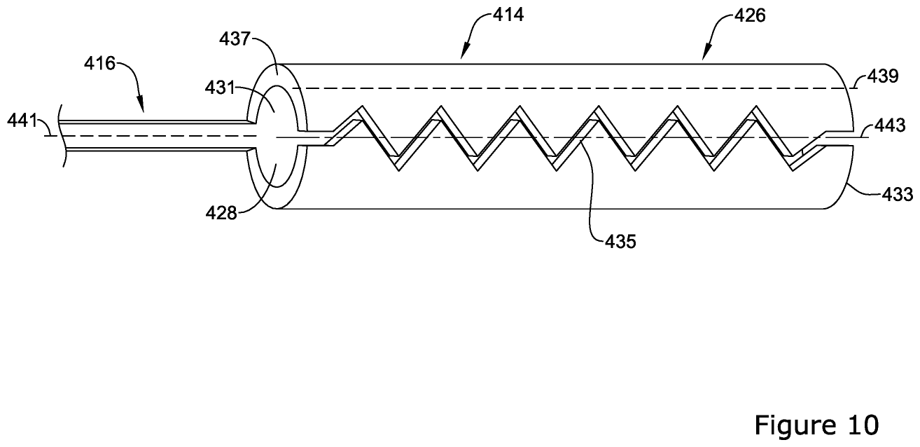

FIG. 10 shows a partial perspective view of another example embodiment of a guide extension catheter 414. The guide extension catheter 414 may be similar in form and function to other embodiments described herein, and may include structure and/or materials that are the same as or similar to other embodiments described herein. For example, similar to other embodiments discussed herein, the guide extension catheter 414 includes a proximal shaft 416, a distal sheath 426, a sheath wall 437, a lumen 428, a proximal opening 431, a distal opening 433, a proximal shaft central longitudinal axis 441, a distal sheath and/or lumen central longitudinal axis 443, a longitudinally extending wall axis 439, and an exchange channel 435 defined in and extending through a sheath wall 437 and configured for allowing access (e.g. side access) to the lumen 428 there through. There are no slots defined in the distal sheath 426, but it is contemplated that slots similar to any of those disclosed herein may be included in the distal sheath 426.

As can be appreciated, this embodiment also includes an example of an exchange channel 435 that extends along a path that is indirect along at least a portion of its length, as generally discussed above. In this particular embodiment, when viewed from the side, the exchange channel 435 includes a plurality of sections that are angled relative to one another, and may be described as following a generally zigzag path as it extends along the distal sheath 426 from the proximal opening 431 to the distal opening 433. As may appreciated, the zigzag shape may form one or more sharp and/or hard angles along the pathway, however in other embodiments, the angles may be soft and/or curved. This exchange channel 435 may be considered to extend in an indirect, or non-straight manner as it extends from the proximal opening 431 to the distal opening 433. For example, the exchange channel 435, or a portion thereof, may be characterized as being non-parallel to the central longitudinal axis 443 and/or non-parallel to the longitudinally extending wall axis 439.

The exchange channel 435 is configured for allowing access (e.g. side access) into the lumen 428 by a medical device. The guide extension catheter 414 can thus be used in a similar manner and/or method to that discussed herein, for example, with reference to FIGS. 3-6, wherein a portion of a medical device 15 may be passed through the exchange channel 435 and into the lumen 428, and the guide extension catheter 414 can be advanced into a guide catheter 10 and over the medical device 15. The medical device 15 simply needs to be passed through the indirect and/or non-straight (e.g. zigzag) exchange channel 435.

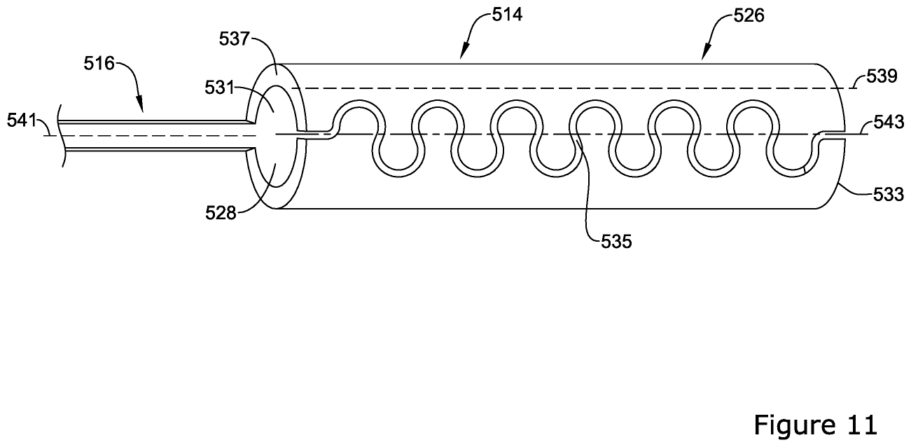

FIG. 11 shows a partial perspective view of another example embodiment of a guide extension catheter 514. The guide extension catheter 514 may be similar in form and function to other embodiments described herein, and may include structure and/or materials that are the same as or similar to other embodiments described herein. For example, the guide extension catheter 514 includes a proximal shaft 516, a distal sheath 526, a sheath wall 537, a lumen 528, a proximal opening 531, a distal opening 533, a proximal shaft central longitudinal axis 541, a distal sheath and/or lumen central longitudinal axis 543, a longitudinally extending wall axis 539, and an exchange channel 535 defined in and extending through a sheath wall 537 and configured for allowing access (e.g. side access) to the lumen 528 there through. There are no slots defined in the distal sheath 526, but it is contemplated that slots similar to any of those disclosed herein may be included in the distal sheath 526.

As can be appreciated, this embodiment also includes an example of an exchange channel 535 that extends along a path that is indirect along at least a portion of its length, as generally discussed above. In this particular embodiment, when viewed from the side, the exchange channel 535 follows a path that is generally continuously curved and/or serpentine and/or sinuous shaped as it extends along the distal sheath 526 from the proximal opening 531 to the distal opening 533. This exchange channel 535 may be considered to extend in an indirect, or non-straight manner as it extends from the proximal opening 531 to the distal opening 533. For example, the exchange channel 535, or a portion thereof, may characterized as being non-parallel to the central longitudinal axis 543 and/or non-parallel to the longitudinally extending wall axis 539. In this embodiment, the exchange channel 535 is constantly curved rather than forming sharp and/or hard angles. It may also be appreciated that the exchange channel 535 forms a path through the distal sheath 526 from the proximal opening 531 to the distal opening 533 that allows the remaining structure of the distal sheath 526 to be interlocking in nature. For example, the pathway of the exchange channel 535 forms skinny neck portions and larger head portions in the remaining structure of the distal sheath 526 that are interlocking and/or zipper-like in nature. Such structure may prevent and/or make it more difficult for the distal sheath 526 to be splayed open.

The exchange channel 535 is configured for allowing access (e.g. side access) into the lumen 528 by a medical device. The guide extension catheter 514 can thus be used in a similar manner and/or method to that discussed herein, for example, with reference to FIGS. 3-6, wherein a portion of a medical device 15 may be passed through the exchange channel 535 and into the lumen 528, and the guide extension catheter 514 can be advanced into a guide catheter 10 and over the medical device 15. The medical device 15 simply needs to be passed through the non-straight (e.g. serpentine and/or sinuous) exchange channel 535.

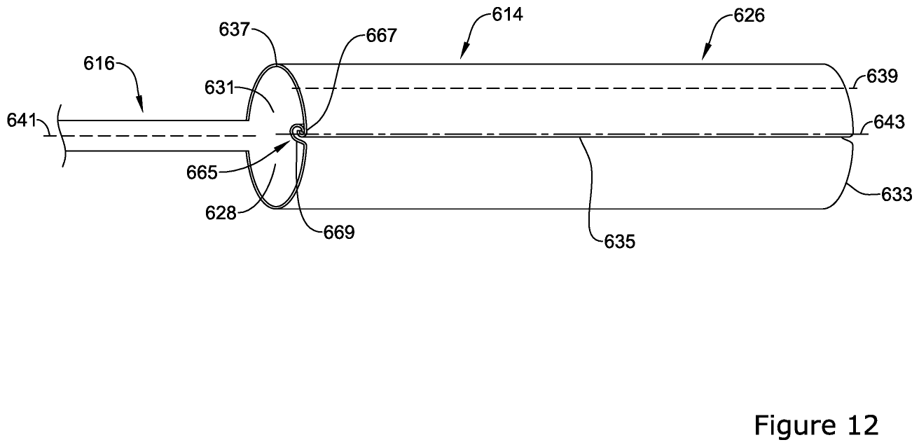

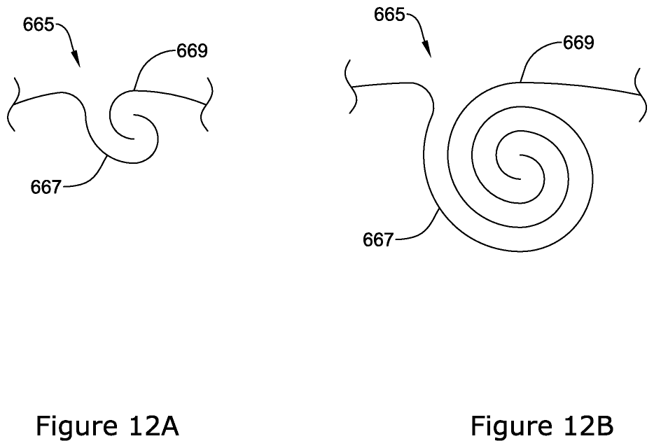

FIG. 12 shows a partial perspective view of another example embodiment of a guide extension catheter 614. The guide extension catheter 614 may be similar in form and function to other embodiments described herein, and may include structure and/or materials that are the same as or similar to other embodiments described herein. For example, the guide extension catheter 614 includes a proximal shaft 616, a distal sheath 626, a sheath wall 637, a lumen 628, a proximal opening 631, a distal opening 633, a proximal shaft central longitudinal axis 641, a distal sheath and/or lumen central longitudinal axis 643, a longitudinally extending wall axis 639, and an exchange channel 635 defined in and extending through a sheath wall 637 and configured for allowing access (e.g. side access) to the lumen 628 there through. There are no slots defined in the distal sheath 626, but it is contemplated that slots similar to any of those disclosed herein may be included in the distal sheath 626.

This is another example embodiment where the exchange channel 635 extends along a direct straight line and/or straight path along its length from the proximal opening 631 (e.g. at the proximal end of the sheath) to the distal opening 633 (e.g. at the distal end of the sheath). For example, the exchange channel 635 may extend parallel to the central longitudinal axis 643 and/or along or parallel to the longitudinally extending wall axis 639. However, in other embodiments, the exchange channel 635 may be configured to extend along a path that is indirect along at least a portion of its length, for example, as in other embodiments disclosed herein.

This embodiment includes a closing structure 665 for selectively closing the exchange channel 635. In particular, the distal sheath 626 defines overlapping portions 667 and 669 that extend along the exchange channel 635 that may include engagement and/or interlocking structures, and may be shaped and/or configured to engage each other to selectively and/or releasably close the exchange channel 635. The overlapping portions 667 and 669 may simply overlap each other, or may overlap in a manner in which they may interlock. For example, the overlapping portions 667 and 669 from either side of the exchange channel 635 may be rolled and/or curved and/or wrapped about each other to define interlocking edges and/or channels as show in the cross-sectional view of FIG. 12A. Additionally and/or alternatively, the overlapping portions 667 and 669 may define a plurality of wraps and/or rolls in a spiral like fashion, for example, as show in the cross-sectional view of FIG. 12B.

The exchange channel 635 may be configured for allowing access (e.g. side access) into the lumen 628 by a medical device, for example when opened. The guide extension catheter 614 can thus be used in a similar manner and/or method to that discussed herein, for example, with reference to FIGS. 3-6, wherein a portion of a medical device 15 may be passed through the exchange channel 635 and into the lumen 628, and the guide extension catheter 514 can be advanced into a guide catheter 10 and over the medical device 15. In this particular embodiment, however, the exchange channel 635 may need to be opened prior to and during insertion of the medical device 15, for example by opening and/or disengaging the overlapping portions 667 and 669, and may be selectively and/or releasably closed by closing and/or engaging the overlapping portions 667 and 669 after insertion of the medical device 15 there through.

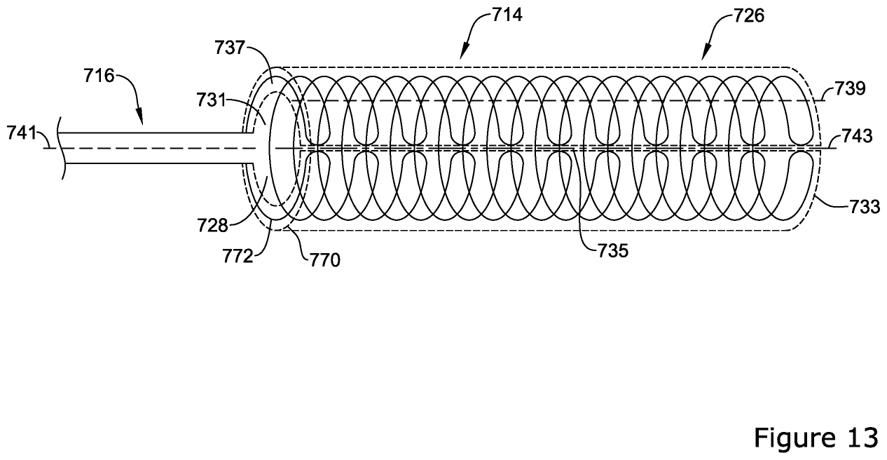

FIG. 13 shows a partial perspective view of another example embodiment of a guide extension catheter 714. The guide extension catheter 714 may be similar in form and function to other embodiments described herein, and may include structure and/or materials that are the same as or similar to other embodiments described herein. For example, the guide extension catheter 714 includes a proximal shaft 716, a distal sheath 726, a sheath wall 737, a lumen 728, a proximal opening 731, a distal opening 733, a proximal shaft central longitudinal axis 741, a distal sheath and/or lumen central longitudinal axis 743, a longitudinally extending wall axis 739, and an exchange channel 735 defined in and extending through a sheath wall 737 and configured for allowing access (e.g. side access) to the lumen 728 there through. There are no slots defined in the distal sheath 726, but it is contemplated that slots similar to any of those disclosed herein may be included in the distal sheath 726.

This is another example embodiment where the exchange channel 735 extends along a direct straight line and/or straight path along its length from the proximal opening 731 (e.g. at the proximal end of the sheath) to the distal opening 733 (e.g. at the distal end of the sheath). For example, the exchange channel 735 may extend parallel to the central longitudinal axis 743 and/or along or parallel to the longitudinally extending wall axis 739. However, in other embodiments, the exchange channel 735 may be configured to extend along a path that is indirect along at least a portion of its length, for example, as in other embodiments disclosed herein

In this embodiment, the distal sheath 714 may include or be made of a plurality of structures and/or materials. For example, the distal sheath 726 may include a reinforcement 772 and a casing 770. The reinforcement 772 may include and/or be a winding, braid, coil, mesh, or the like that is disposed within the casing 770. The reinforcement member 772 and casing 770 may be made of any suitable material as discussed herein. For one example, the reinforcement 772 may be a metallic material disposed within a polymer casing 770.

In this particular embodiment, the reinforcement 772 may be made with a winding of material making a wire frame that defines a lengthwise or longitudinal slit and/or opening in which the exchange channel 735 may be defined. In some embodiments, such a reinforcement 772 structure may be made by a single winding of material that wound in a somewhat modified coil and/or interrupted coil and/or undulating coil like manner to define the reinforcing member 772 with the lengthwise slit. In other words, the single winding may be wound in such manner that it doubles-back on itself so as to form the longitudinal slit. In other embodiments, a braid and/or knit pattern that may include one or a plurality of windings may be used to define a reinforcing structure 772 having a lengthwise slit. For example, three dimensional braiding technology may be used to create such a structure. The casing 770 may then be disposed on, over, and/or around the reinforcement 772, and the exchange channel 735 is disposed and/or defined within the slit of the reinforcement 772 to allow access (e.g. side access) to the lumen 728.

In yet other embodiments, the distal sheath 726 may include and/or be made of a winding, braid, coil, mesh, or the like, similar to the reinforcement 772 discussed above, but such structures may be used as the sheath 726 without the casing 770. In other words, such winding, braid, coil, mesh, or like structures may alone form the distal sheath 726 defining the exchange channel 735 (e.g. without a casing).

The exchange channel 735 may be configured for allowing access (e.g. side access) into the lumen 728 by a medical device, for example when opened. The guide extension catheter 714 can thus be used in a similar manner and/or method to that discussed herein, for example, with reference to FIGS. 3-6, wherein a portion of a medical device 15 may be passed through the exchange channel 735 and into the lumen 728, and the guide extension catheter 714 can be advanced into a guide catheter 10 and over the medical device 15.

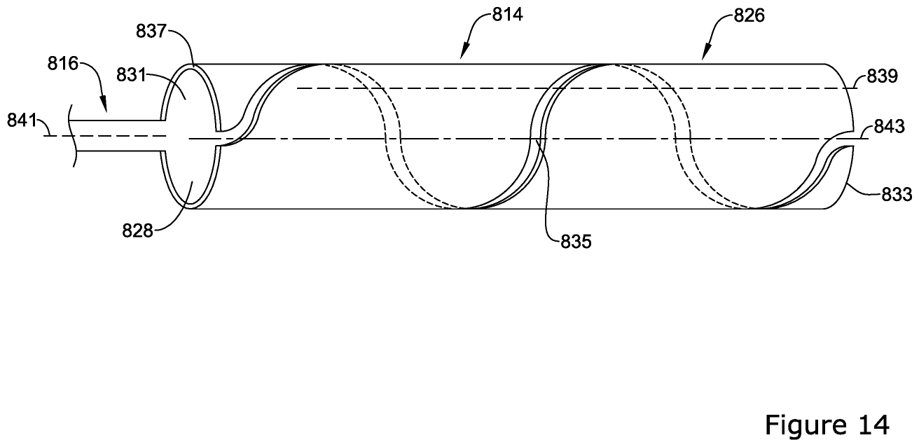

FIG. 14 shows a partial perspective view of another example embodiment of a guide extension catheter 814. The guide extension catheter 814 may be similar in form and function to other embodiments described herein, and may include structure and/or materials that are the same as or similar to other embodiments described herein. For example, similar to other embodiments discussed herein, the guide extension catheter 814 includes a proximal shaft 816, a distal sheath 826, a sheath wall 837, a lumen 828, a proximal opening 831, a distal opening 833, a proximal shaft central longitudinal axis 841, a distal sheath and/or lumen central longitudinal axis 843, a longitudinally extending wall axis 839, and an exchange channel 835 defined in and extending through a sheath wall 837 and configured for allowing access (e.g. side access) to the lumen 828 there through. There are no slots defined in the distal sheath 826, but it is contemplated that slots similar to any of those disclosed herein may be included in the distal sheath 826.

As can be appreciated, this embodiment also includes an example of an exchange channel 835 that extends along a path that is indirect along at least a portion of its length, as generally discussed above. In this particular embodiment, when viewed from the side, the exchange channel 835 follows a path that is generally helically and/or spirally disposed about and/or around the distal sheath 826 (e.g. helically disposed about the axis 843) as it extends from the proximal opening 831 to the distal opening 833. This exchange channel 835 may be considered to extend in an indirect, or non-straight manner as it extends from the proximal opening 831 to the distal opening 833. For example, the exchange channel 835, or a portion thereof, may characterized as being non-parallel to the central longitudinal axis 843 and/or non-parallel to the longitudinally extending wall axis 839.

The exchange channel 835 is configured for allowing access (e.g. side access) into the lumen 828 by a medical device. The guide extension catheter 814 can thus be used in a similar manner and/or method to that discussed herein, for example, with reference to FIGS. 3-6, wherein a portion of a medical device 15 may be passed through the exchange channel 835 and into the lumen 828, and the guide extension catheter 814 can be advanced into a guide catheter 10 and over the medical device 15. The medical device 15 simply needs to be passed through the non-straight (e.g. helical) exchange channel 835.

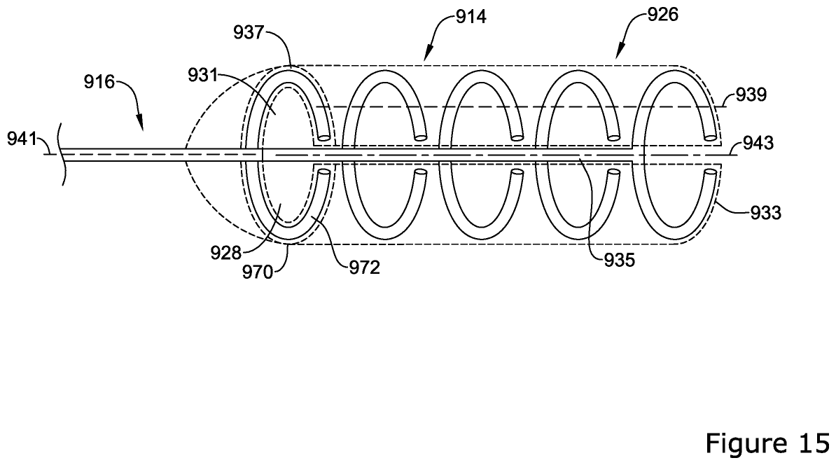

FIG. 15 shows a partial perspective view of another example embodiment of a guide extension catheter 914. The guide extension catheter 914 may be similar in form and function to other embodiments described herein, and may include structure and/or materials that are the same as or similar to other embodiments described herein. For example, the guide extension catheter 914 includes a proximal shaft 916, a distal sheath 926, a sheath wall 937, a lumen 928, a proximal opening 931, a distal opening 933, a proximal shaft central longitudinal axis 941, a distal sheath and/or lumen central longitudinal axis 943, a longitudinally extending wall axis 939, and an exchange channel 935 defined in and extending through a sheath wall 937 and configured for allowing access (e.g. side access) to the lumen 928 there through. There are no slots defined in the distal sheath 926, but it is contemplated that slots similar to any of those disclosed herein may be included in the distal sheath.

This is another example embodiment where the exchange channel 935 extends along a direct straight line and/or straight path along its length from the proximal opening 931 (e.g. at the proximal end of the sheath) to the distal opening 933 (e.g. at the distal end of the sheath). For example, the exchange channel 935 may extend parallel to the central longitudinal axis 943 and/or along or parallel to the longitudinally extending wall axis 939. However, in other embodiments, the exchange channel 935 may be configured to extend along a path that is indirect along at least a portion of its length, for example, as in other embodiments disclosed herein.

This is also another example embodiment where the distal sheath 926 may include or be made of a plurality of structures and/or materials. For example, the distal sheath 926 may include a reinforcing structure 972 and a casing 970. The reinforcing structure 972 may include a plurality of ribs or supports that may be generally open ring and/or “C-shaped”, either with or without a backbone like structure interconnecting the ribs or supports. The ribs or supports of the reinforcing structure 972 may be aligned such that a slit and/or channel is defined there along, in which the exchange channel 935 may be defined. The casing 970 may be disposed on, over, and/or around the reinforcing structure 972, with the exchange channel 935 defined in the slit of the reinforcing structure 972. The reinforcing structure 972 and casing 970 may be made of any suitable material as discussed herein. For one example, the reinforcing structure 972 may be a metallic material disposed within a polymer casing 970.

The exchange channel 935 is configured for allowing access (e.g. side access) into the lumen 928 by a medical device. The guide extension catheter 914 can thus be used in a similar manner and/or method to that discussed herein, for example, with reference to FIGS. 3-6, wherein a portion of a medical device 15 may be passed through the exchange channel 935 and into the lumen 928, and the guide extension catheter 914 can be advanced into a guide catheter 10 and over the medical device 15.

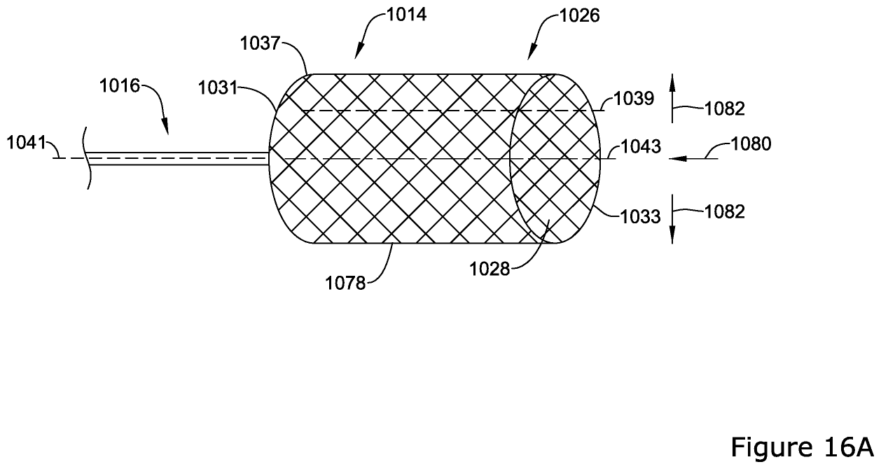

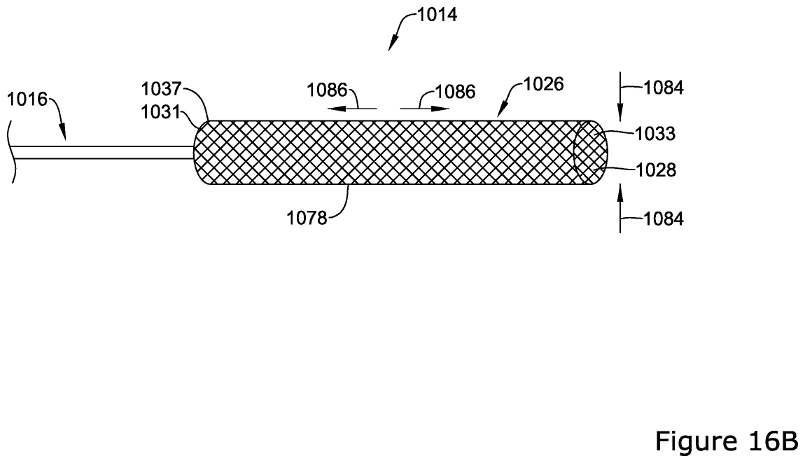

FIGS. 16A and 16B shows a partial perspective views of another example embodiment of a guide extension catheter 1014, which may be somewhat similar in some aspects to the form and function to the other guide extension catheters discussed herein, but may include a distal sheath 1026 that is configured to include a first, radially expanded configuration, for example as shown in FIG. 16A, and a second radially contracted and/or non-expanded configuration, for example as shown in FIG. 16B.

As discussed herein, it may be difficult or impossible to thread a typical guide extension catheter over and/or onto the very proximal end of the medical device. For example, such medical devices often include a hub and/or manifold disposed at the proximal end thereof, and a typical guide extension catheter may not fit there over. Additionally, even if no such hub and/or manifold exists, threading the proximal end of a medical device into a small diameter sheath of a guide extension catheter may be a challenge. As discussed herein, it would be desirable to provide a guide extension catheter that includes structure and/or a mechanism and/or means for allowing the guide extension catheter to be introduced into the guide catheter and/or around or over the medical device after the medical device has already been introduced into the guide catheter and/or into the anatomy during a procedure. In this embodiment, the structure and/or a mechanism and/or means for allowing this includes providing the distal sheath 1026 with the first, radially expanded configuration (FIG. 16A), which can be used to facilitate loading the distal sheath 1026 over and/or around the proximal end of the medical device, and a second, radially contracted and/or non-expanded configuration (FIG. 16B), which can be used to facilitate advancing the guide extension catheter 1014 into the guide catheter over the medical device for use.

Similar to other embodiments discussed herein, the guide extension catheter 1014 includes a proximal shaft 1016, a distal sheath 1026, a sheath wall 1037, a lumen 1028, a proximal opening 1031, a distal opening 1033, a proximal shaft central longitudinal axis 1041, a distal sheath and/or lumen central longitudinal axis 1043, and a longitudinally extending wall axis 1039. In this embodiment, the distal sheath 1026 may include and/or be made of a tubular braided structure 1078. The distal sheath 1026 and/or braided structure 1078 may be configured to include the first, radially expanded configuration, for example as shown in FIG. 16A, and the second radially contracted and/or non-expanded configuration, for example as shown in FIG. 16B. The braided structure 1078 may include a lengthwise pitch such that lengthwise compression of the distal sheath 1026 and/or braided structure 1078 (represented by arrow 1080) may facilitate radial expansion (represented by arrows 1082) thereby resulting in an increase in the diameter of the distal sheath 1026 and/or increase in the diameter of the lumen 1028, as shown in FIG. 16A. In this radially expanded configuration, a proximal end of a medical device may be loaded through the lumen 1028 such that the distal sheath 1026 is disposed on and/or over the medical device. As shown in FIG. 16B, lengthwise expansion of the distal sheath 1026 and/or braided structure 1078 (represented by arrows 1086) may facilitate radial contraction (represented by arrows 1084) thereby resulting in a decrease in the diameter of the distal sheath 1026 and/or decrease in the diameter of the lumen 1028. In this radially contracted and/or non-expanded configuration, the distal sheath 1026 may be advanced into the guide catheter over the medical device. It is noted that in this particular embodiment, there is not an exchange channel defined in the distal sheath 1026. However, it is contemplated that in other embodiments, an exchange channel may be included, if so desired. The materials that may be used for the various structures in this embodiment may be the same as or similar to those materials described herein.

The guide extension catheter 1014 can be used in a somewhat similar manner and/or method as described herein, for example with reference to FIGS. 4-6, but alternatively to using an exchange channel (e.g. side access) to dispose the medical device within the lumen 1028, the expanded configuration of the distal sheath 1026 may be used to dispose the medical device within the lumen 1028. For example, an example method of using the guide extension catheter 1014 includes use in conjunction with a guide catheter 10 and a medical device 15, wherein the medical device 15 includes a portion that is already disposed within the guide catheter 10 (as shown in FIG. 4.). In particular, as shown, the medical device 10 includes a distal region 45 disposed within the guide catheter 10 and a proximal region 47 including a proximal end disposed outside of the guide catheter 10. The guide extension catheter 1014 in this embodiment (as shown in FIGS. 16A and 16B) includes the proximal shaft 1016 and the distal sheath 1026 defining the lumen 1028. The distal sheath 1026 in this embodiment includes the first configuration (e.g. FIG. 16A) that is radially expanded and the second configuration (e.g. FIG. 16B) that is radially contracted and/or non-expanded. The method includes disposing the distal sheath 1026 into the first configuration; passing the proximal end and a portion of the proximal region 47 of the medical device 15 into the lumen 1028 and through the distal sheath 1026 such that the distal sheath 1026 is disposed over the proximal region 47 of the medical device 15; shifting the distal sheath 1026 into the second radially contracted and/or non-expanded configuration on/over the medical device 15; and advancing the guide extension catheter 1014 into the guide catheter 10 over the medical device 15.

The materials that can be used for the various components of the guide extension catheters 14, 114, 214, 314, 414, 514, 614, 714, 814, 914, and/or 1014 (and/or other guide extension catheters disclosed herein) and the various devices disclosed herein may include those commonly associated with medical devices. For simplicity purposes, the following discussion makes reference to proximal member, the distal sheath and/or other components of the guide extension catheter. However, this is not intended to limit the devices and methods described herein, as the discussion may be applied to other similar members and/or components of members or devices disclosed herein.

The proximal member, the distal sheath and/or other components of the guide extension catheter may be made from a metal, metal alloy, polymer (some examples of which are disclosed below), a metal-polymer composite, ceramics, combinations thereof, and the like, or other suitable material or composites of materials. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, 316LV, and 17-7 stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

The proximal member, the distal sheath and/or other components of the guide extension catheter may include the use of different metals or polymers with differing stiffness or durometers along the length thereof. For example, a more proximal section of the distal sheath may include a metal or polymer with higher stiffness or durometer and a more distal section may include a metal or polymer with lower stiffness or durometer. The proximal member, the distal sheath and/or other components of the guide extension catheter may include or be made of one or more layers of materials. For example, a plurality or layers of differing material may be used to form all or portions of the distal sheath. The distal sheath may also include and/or be entirely made of a reinforcement member, such as a braid, coil, mesh, or the like.

In at least some embodiments, portions or all of the guide extension catheter may be doped with, made of, or otherwise include a radiopaque material. Radiopaque materials are understood to be materials capable of producing a relatively bright image on a fluoroscopy screen or another imaging technique during a medical procedure. This relatively bright image aids the user of the guide extension catheter in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the guide extension catheter to achieve the same result.

In some embodiments, a degree of Magnetic Resonance Imaging (MRI) compatibility is imparted into the guide extension catheter. For example, the guide extension catheter, or portions thereof, may be made of a material that does not substantially distort the image and create substantial artifacts (e.g., gaps in the image). Certain ferromagnetic materials, for example, may not be suitable because they may create artifacts in an MRI image. The guide extension catheter, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method of using a guide extension catheter in conjunction with a guide catheter and a medical device that includes a distal region disposed within the guide catheter and a proximal region disposed outside of the guide catheter, the guide extension catheter including a proximal shaft and a distal sheath defining a lumen, the distal sheath including an exchange channel allowing for access to the lumen, the method comprising:
passing a portion of the proximal region of the medical device through the exchange channel and into the lumen; and
advancing the guide extension catheter into the guide catheter over the medical device.

2. The method of claim 1, wherein the guide catheter is disposed within an anatomy of a patient, and advancing the guide extension catheter into the guide catheter over the medical device includes advancing the guide extension catheter into the anatomy of the patient without removing the distal region of the medical device from the guide catheter.

3. The method of claim 1, wherein the guide catheter is disposed within an anatomy of a patient, and advancing the guide extension catheter into the guide catheter over the medical device includes advancing the guide extension catheter at least partially into a coronary artery.

4. The method of claim 1, wherein the distal sheath defines a proximal lumen opening and a distal lumen opening, and the exchange channel extend from the proximal lumen opening to the distal lumen opening.

5. The method of claim 4, wherein the exchange channel extends from the proximal lumen opening to the distal lumen opening along a direct straight line.

6. The method of claim 4, wherein the exchange channel extends from the proximal lumen opening to the distal lumen opening along a path that is indirect.

7. The method of claim 6, wherein the indirect path includes at least a portion that is angled, curved, serpentine, sinuous, undulating, winding, slanted, spiral, helical, zigzag, or sine wave shaped.

8. The method of claim 1, wherein the distal sheath includes a plurality of slots formed therein.

9. The method of claim 1, wherein the distal sheath includes closing structures to selectively close the exchange channel.

10. A guide extension catheter, comprising:
a proximal shaft having a distal end region;
a distal sheath coupled to the distal end region, the distal sheath including a sheath wall defining a lumen, a proximal lumen opening, a distal lumen opening, and an exchange channel extending through the sheath wall;
the exchange channel extending from the proximal lumen opening to the distal lumen opening, and being designed to allow an access region of a medical device to pass there through and into the lumen, so that the distal sheath can be advanced over the medical device; and
wherein the exchange channel extends from the proximal lumen opening to the distal lumen opening along an indirect path.

11. The guide extension catheter of claim 10, wherein the distal sheath defines a central longitudinal axis, and the sheath wall defines a wall axis that extends between a proximal end of the distal sheath and a distal end of the distal sheath, the wall axis being parallel to the central longitudinal axis; and
wherein at least a portion of the exchange channel is non-parallel with the wall axis.

12. The method of claim 10, wherein the exchange channel includes at least a portion that is angled, curved, serpentine, sinuous, undulating, winding, slanted, spiral, helical, zigzag, or sine wave shaped.

13. The guide extension catheter of claim 10, wherein the distal sheath has a plurality of slots formed therein.

14. The guide extension catheter of claim 10, wherein the distal sheath includes closing structures to selectively close the exchange channel.

15. The guide extension catheter of claim 10, wherein the distal sheath includes a reinforcing structure.

16. A guide extension catheter, comprising:
a proximal shaft having a distal end region;
a distal sheath coupled to the distal end region, the distal sheath having a proximal end and a distal end, defining a lumen extending there through, and having an exchange channel formed therein, the exchange channel extending from the proximal end to the distal end and being designed to allow an access region of a medical device to pass there through and into the lumen so that the distal sheath can be advanced over the medical device; and
the distal sheath includes a plurality of slots formed therein.

17. The guide extension catheter of claim 16, wherein the exchange channel extends from the proximal end to the distal end along a direct straight line.

18. The guide extension catheter of claim 16, wherein the exchange channel extends from the proximal end to the distal end along an indirect path.

19. The guide extension catheter of claim 18, wherein the indirect path includes at least a portion that is angled, curved, serpentine, sinuous, undulating, winding, slanted, spiral, helical, zigzag, or sine wave shaped.

20. The guide extension catheter of claim 16, wherein the proximal shaft has a proximal shaft outer diameter and the distal sheath has a distal sheath outer diameter, and the proximal shaft outer diameter is less than the distal sheath outer diameter.
